(19) United States
(12) Patent Application Publication
(10) Pub. No.: US 2008/0119840 A1
(43) Pub. Date: May 22, 2008

(54) Ridged Cryoprobe Tip

(76) Inventors: David W. Vancelette, San Diego, CA (US); Michael W.V. Perkins, Minnetonka, MN (US)

Correspondence Address: AMS RESEARCH CORPORATION, 10700 BREN ROAD WEST, MINNETONKA, MN, 55343, US

(21) Appl. No.: 11/943,777

(22) Filed: Nov. 21, 2007

Related U.S. Application Data
(60) Provisional application No. 60/866,628, filed on Nov. 21, 2006.

(51) Int. Cl. A61B 18/18

(52) U.S. Cl. 606/23; 606/20; 606/21

(57) ABSTRACT

A cryoprobe for use in a cryosurgical system in which the probe tip has an increased surface area by having a corrugated, waved, or otherwise ridged configuration in its inner and outer surfaces. The increased surface area functions to provide greater surface area on the probe tip, and thus greater heat transfer, between the refrigerant and inner surface of the cryoprobe and between the outer surface of the cryoprobe and the target tissue.

### PRIORITY CLAIM

The present application claims priority to U.S. Provisional Application Ser. No. 60/866,628, filed Nov. 21, 2006 and entitled “RIDGED CRYOPROBE TIP”, which is herein incorporated by reference in its entirety.

### FIELD OF THE INVENTION

The present disclosure relates to cryosurgical probes for use in cryosurgical procedures such as in the treatment of benign or cancerous tissues. More particularly the present invention relates to cryoprobe designs to improve heat transfer during cryosurgical procedures.

### BACKGROUND OF THE INVENTION

Cryosurgical probes are used to treat a variety of diseases. Cryosurgical probes quickly freeze diseased body tissue, causing the tissue to die after which it will be absorbed by the body, expelled by the body, sloughed off or replaced by scar tissue. Cryothermal treatment can be used in cancer treatments such as prostate, breast and liver cancer. Cryosurgery also has a variety of gynecological applications and may be used for the treatment of a number of other diseases and conditions including, but certainly not limited to, benign prostate disease, renal cancer, glaucoma and other eye diseases.

A variety of cryosurgical instruments variously referred to as cryoprobes, cryosurgical probes, cryosurgical ablation devices, cryostats and cryocoolers have been used for cryosurgery. These devices typically use the principle of Joule-Thomson expansion to generate cooling. They take advantage of the fact that most fluids, when rapidly expanded, become extremely cold. In these devices, a high pressure gas mixture is expanded through a nozzle inside a small cylindrical shaft or sheath typically made of steel. The Joule-Thomson expansion cools the steel sheath to a cold temperature very rapidly. The cryosurgical probes then form ice balls which freeze diseased tissue. A properly performed cryosurgical procedure allows cryoablation of the diseased tissue without undue destruction of surrounding healthy tissue.

### SUMMARY OF THE INVENTION

The present disclosure is directed to a cryoprobe for use in a cryosurgical system in which the probe tip has corrugated, waved, or otherwise ridged folds in its outer and/or inner surfaces. The various shapes function to provide greater surface area on the probe tip, and thus greater heat transfer, between the refrigerant and inner surface of the cryoprobe and between the outer surface of the cryoprobe and the target tissue.

In one aspect of the present disclosure, a cryosurgical probe can include a probe tip comprising an elongated tube portion and an end piece. The elongated tube portion of cryoprobe tip can have ridged folds on its outer and/or inner surfaces. In some representative embodiments, an end piece of probe tip is provided with a pointed distal end or trocar configuration to allow the cryoprobe tip to penetrate tissue.

In another aspect of the present disclosure, a method of performing cryosurgery comprises using a cryoprobe having a tip portion with a ridged inner and/or outer surface so as to promote better heat transfer performance between the cryoprobe and targeted tissue. In some representative embodiments, the tip portion of the cryoprobe tip is provided with a piercing end so as to allow the cryoprobe tip to selectively penetrate surface tissue. Cryosurgery can then be performed on targeted interior tissue with the ridged outer surface of cryoprobe tip contacting the tissue.

In yet another aspect of the present disclosure, a cryosurgical treatment system can comprise a closed loop cryosurgical system. The cryosurgical system can include a plurality of cryoprobes used to freeze targeted regions of tissue. Each cryoprobe can have a tip portion with a ridged inner and/or outer surface to better promote heat transfer between a refrigerant circulated in the cryoprobe and the cryoprobe tip and between the cryoprobe and the targeted tissue.

The above summary of the various representative embodiments of the invention is not intended to describe each illustrated embodiment or every implementation of the invention. Rather, the embodiments are chosen and described so that others skilled in the art may appreciate and understand the principles and practices of the invention. The figures in the detailed description that follows more particularly exemplify these embodiments.

### BRIEF DESCRIPTION OF THE FIGURES

These as well as other objects and advantages of this invention, will be more completely understood and appreciated by referring to the following more detailed description of the presently preferred exemplary embodiments of the invention in conjunction with the accompanying drawings of which:

FIG. 1 is a side view of an embodiment of a cryoprobe tip according to the present disclosure.

FIG. 2 is a section view of the cryoprobe tip of FIG. 1 taken at line 2-2 of FIG. 1.

FIG. 3A is a section view of an embodiment of a cryoprobe tip according to the present disclosure.

FIG. 3B is a section view of an embodiment of a cryoprobe tip according to the present disclosure.

FIG. 4A is a side view of an embodiment of a sheet of tip material that can be used to form a portion of a cryoprobe tip according to the present disclosure.

FIG. 4B is a perspective end view of an embodiment of a cryoprobe tip formed with the tip material of FIG. 4A.

FIG. 5 is a view a closed loop cryosurgical system with which a cryoprobe according to the present disclosure may be used.

### DETAILED DESCRIPTION

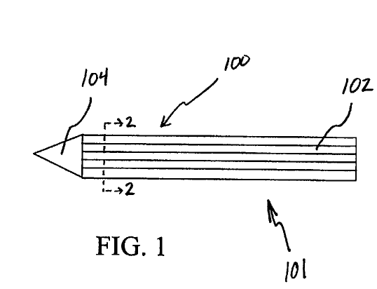

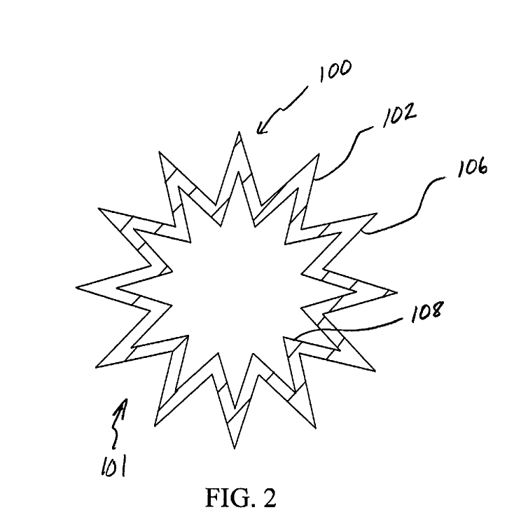

Referring to FIGS. 1 and 2, there can be seen a tip portion 100 of a cryoprobe 101 for use in a cryosurgical system according to an embodiment of the present invention. The tip portion 100 constitutes the region of the cryoprobe 101 that performs the actual cryogenic treatment. Generally, the cryogenic treatment conducted with cryoprobe 100 includes freezing and removing selected portions of tissue at a treatment site such as, for example, cancerous tissue. Tip portion 100 generally comprises an elongated tube portion 102 and an end piece 104. The cryoprobe 101 is typically constructed such that the outer diameter of the cryoprobe tip 100 is from about 1.4 to about 5.0 mm, depending on the cryosurgical application for which the cryoprobe 101 is being used.

As illustrated in FIG. 2, the elongated tube portion 102 generally comprises an outer surface 106 and an inner surface 108 wherein one or both of outer surface 106 and inner surface 108 can be corrugated, wavelike or otherwise ridged. By forming the elongated tube portion 102 and more specifically, outer surface 106 and/or inner surface 108 with the corrugated, waved or ridged configuration, the heat transfer properties between a refrigerant expanded within the tip portion 100 and the inner surface 108 is improved. A corrugated, wavelike or ridged design increases the available surface area across which convection occurs, allowing the refrigerant to more quickly and effectively cool the cryoprobe tip 100. The corrugated, wavelike or ridged design also enhances heat transfer between the outer surface 106 of the tip portion 100 and the selected target tissue. With the corrugated, wavelike or ridged design, a greater surface area of tip portion 100 is able to contact the target tissue, thereby cooling the tissue more quickly and efficiently. In addition, the corrugated, wavelike or ridged design also decreases damage to peripheral, healthy tissue surrounding the target tissue because the surface area at the outermost portion of the tip portion's diameter is reduced.

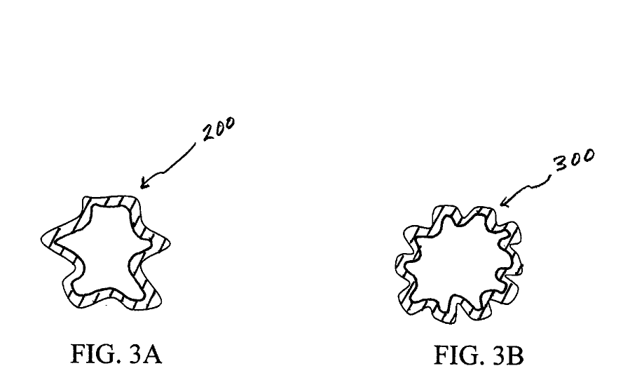

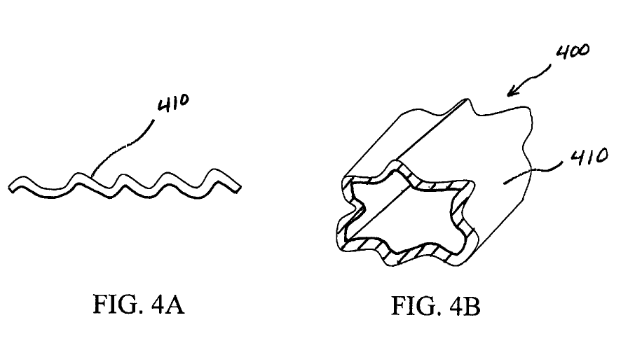

Cryoprobe 101 and more specifically, tip portion 100 can be fabricated in a variety of ways such as, for example, through suitable extrusion or molding techniques such that elongated tube portion 102 includes corrugations, waves, or ridges in a wide variety of shapes, sizes, and configurations. For instance, FIGS. 3A and 3B depict two alternative designs for cryoprobe 101 wherein elongated tube portion 102 is extruded to form a tube profile 200 or tube profile 300 respectively. Alternatively, cryoprobe 101, and more specifically, elongated tube portion 102 can be formed from a waved sheet 410 as illustrated in FIG. 4A. Referring to FIG. 4B, waved sheet 410, can be rolled into a cylinder and joined using a suitable joining technique such as, for example, welding or adhesive joining, so as to form a tube profile 400 for elongated tube portion 102. Wire-cut EDM may also be used to form cryoprobe tip. End piece 104 can be integral to the elongated tube portion 102 or in some representative embodiments, end piece 104 can be formed separately and attached to elongated tube portion 102 using a suitable joining technique such as, for example, welding or adhesive joining to form tip portion 100. Tip portion 100 can be formed of suitable surgical grade materials including, for example, stainless steel, nitinol, or any other biocompatible, joinable alloy compatible with the above fabrications processes and application.

In some embodiments of cryoprobe 101, end piece 104 can be fabricated and configured to provide tip portion 100 with a conical, pointed, trocar, or another similar piercing configuration. By configuring end piece 104 to have a piercing feature, a user can penetrate targeted tissue with the end piece 104 so as to guide the cryoprobe tip 100 into the tissue. By piercing the target tissue with end piece 104, the elongated tube portion 102 of tip portion 100 can more easily access internal target tissue. As a result, the enhanced heat transfer due to the corrugated, waved, or ridged outer and/or inner surfaces even causes tissue that is located relatively deeply in the body to be more quickly and effectively treated.

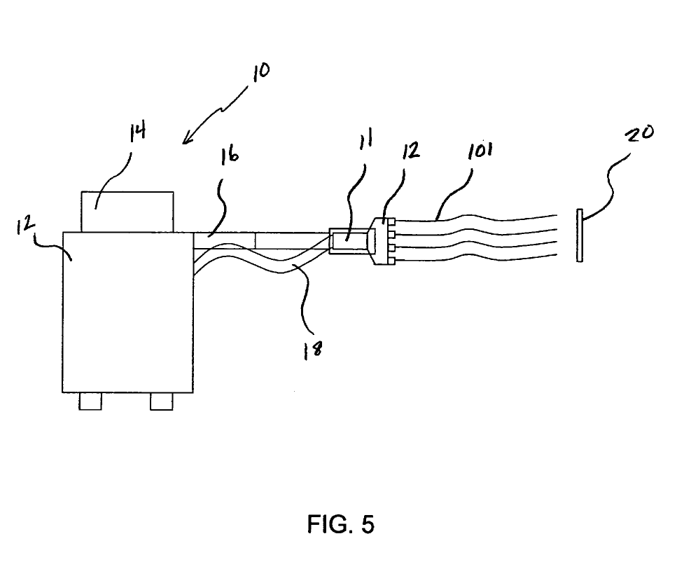

A representative cryoprobe 101 incorporating the various embodiments of tip portion 100 described above can be used in conjunction with various types of cryoablation systems. An example of one such closed loop cryosurgical system 10 is depicted in FIG. 5. Cryosurgical system 10 can include a refrigeration and control console 12 with an attached display 14. Console 12 can contain a primary compressor to provide a primary pressurized, mixed gas refrigerant to the system and a secondary compressor that provides a secondary pressurized, mixed gas refrigerant to the system. The use of mixed gas refrigerants is generally known in the art to provide a dramatic increase in cooling performance over the use of a single gas refrigerant. Console 12 can also include controls that allow for the activation, deactivation, and modification of various system parameters, such as, for example, gas flow rates, pressures, and temperatures of the mixed gas refrigerants. Display 14 provides the operator the ability to monitor, and in some embodiments, adjust the system to ensure it is performing properly and can provide real-time display as well as recording and historical displays of system parameters. One exemplary console that may be used with an embodiment of the present invention is used as part of the Her Option® Office Cryoablation Therapy available from American Medical Systems of Minnetonka, Minn.

With reference to FIG. 5, the high pressure primary refrigerant is transferred to a cryostat heat exchanger module 11 through a flexible line 18. The cryostat heat exchanger module 11 can include a manifold portion 20 that transfers the refrigerant into and receives refrigerant out of one or more cryoprobes 101. The cryostat heat exchanger module 11 and cryoprobes 101 can also be connected to the control console 12 by way of an articulating arm 16, which may be manually or automatically used to position the cryostat heat exchanger module 11 and cryoprobes 101. Although depicted as having the flexible line 18 as a separate component from the articulating arm 16, cryosurgical system 10 may incorporate the flexible line 18 within the articulating arm 16. A positioning grid 20 may be used to properly align and position the cryoprobes 101 for patient insertion.

While the invention has been described in connection with what is presently considered to be the most practical and preferred embodiments, it will be apparent to those of ordinary skill in the art that the invention is not to be limited to the disclosed embodiments. It will be readily apparent to those of ordinary skill in the art that many modifications and equivalent arrangements can be made thereof without departing from the spirit and scope of the present disclosure, such scope to be accorded the broadest interpretation of the appended claims so as to encompass all equivalent structures and products.

1. A cryoprobe for use in a cryosurgical procedure comprising:
an elongated tube having a tip portion, the tip portion having an outer surface and an inner surface, wherein the outer surface and the inner surface define a tube cross-section increasing a tube surface area for contacting tissue.

2. The cryoprobe of claim 1, wherein the tube cross-section defines a corrugated configuration.

3. The cryoprobe of claim 1, wherein the tube cross-section defines a ridged configuration.

4. The cryoprobe of claim 1, wherein the tube cross-section defines a waved configuration.

5. The cryoprobe of claim 1, wherein the tip portion has a trocar configuration.

6. The cryoprobe of claim 1, wherein the elongated tube comprises an extruded, elongated tube.

7. The cryoprobe of claim 1, wherein the elongated tube comprises a rolled sheet material.

8. A closed loop cryosurgical system comprising:
a console having at least one compressor for pressurizing a refrigerant;
a cryostat heat exchanger having a probe manifold; and
a plurality of cryoprobes individually, fluidly connected to the probe manifold, each cryoprobe having a tip portion, the tip portion having an outer surface and an inner surface, wherein the outer surface and the inner surface define a tube cross-section increasing a tube surface area for contacting tissue.

9. The closed loop cryosurgical system of claim 8, wherein the tube cross-section defines a corrugated configuration.

10. The closed loop cryosurgical system of claim 8, wherein the tube cross-section defines a ridged configuration.

11. The closed loop cryosurgical system of claim 8, wherein the tube cross-section defines a waved configuration.

12. The closed loop cryosurgical system of claim 8, wherein the tip portion has a trocar configuration.

13. The closed loop cryosurgical system of claim 8, wherein the elongated tube comprises an extruded, elongated tube.

14. The closed loop cryosurgical system of claim 8, wherein the elongated tube comprises a rolled sheet material.

15. A method of performing a cryosurgical procedure comprising:
increasing a heat transfer area on a cryoprobe by providing a tip portion having a tube cross-section increasing a tube surface area for contacting tissue;
recirculating a refrigerant through the tip portion of the cryoprobe; and
freezing selected tissue with the cryoprobe.

16. The method of claim 15, further comprising:
forming the tip portion in a ridged configuration.

17. The method of claim 15, further comprising:
forming the tip portion in a corrugated configuration.

18. The method of claim 15, further comprising:
forming the tip portion in a waved configuration.

19. The method of claim 15, further comprising:
forming the tip portion in a trocar configuration; and
penetrating tissue with the trocar configuration.
